(19) United States
(12) Patent Application Publication
(10) Pub. No.: US 2025/0375286 A1
(43) Pub. Date: Dec. 11, 2025

(54) TISSUE EXPANDER WITH DRUG DELIVERY CAPABILITY

(71) Applicant: Northwestern University, Evanston, IL (US)

(72) Inventors: John Y. Kim, Lincolnwood, IL (US); Megan E. Fracol, Houston, TX (US)

(21) Appl. No.: 18/737,061

(22) Filed: Jun. 7, 2024

(51) Int. Cl. A61F 2/12

(52) U.S. Cl. CPC A61F 2/12; A61F 2210/009; A61F 2240/001; A61F 2250/0003; A61F 2250/0068

(57) ABSTRACT

An expander and drug delivery device includes a first membrane, where the first membrane is a non-permeable shell that forms an expansion cavity. The device also includes an expansion cavity port that provides access to the expansion cavity, and the non-permeable shell is flexible such that the non-permeable shell inflates in response to insertion of a substance into the expansion cavity via the expansion cavity port. The device includes a second membrane that forms a drug cavity adjacent to the expansion cavity. The device further includes a drug cavity port that connects to the drug cavity and receives a therapeutic solution for delivery to a patient.

### BACKGROUND

Implant-based breast reconstruction is a common reconstructive technique that is used to recreate a breast after mastectomy, accounting for over 75% of breast reconstructions performed in the United States. With breast cancer reconstruction rates rising, over 103,000 women received implant-based reconstruction in 2020 alone. Most often, implant-based reconstruction is performed via a two-stage process, where stage one involves placement of a temporary tissue expander (a temporary implant), followed by placement of a permanent implant months later. The tissue expander is initially inserted in a deflated configuration, and over the course of several months is gradually inflated with either air or saline. This is done to gradually stretch the overlying skin to recreate a breast pocket that will accept the final implant of a desired volume. Inflation occurs via a port that can accept a needle and connect to a central lumen of the tissue expander.

### SUMMARY

An illustrative expander and drug delivery device includes a first membrane, where the first membrane is a non-permeable shell that forms an expansion cavity. The device also includes an expansion cavity port that provides access to the expansion cavity, and the non-permeable shell is flexible such that the non-permeable shell inflates in response to insertion of a substance into the expansion cavity via the expansion cavity port. The device includes a second membrane that forms a drug cavity adjacent to the expansion cavity. The device further includes a drug cavity port that connects to the drug cavity and receives a therapeutic solution for delivery to a patient.

In one embodiment, the drug cavity port connects to the drug cavity by way of a drug cavity port tube such that the drug cavity port is remote from the second membrane. In another embodiment, the drug cavity port is formed on a surface of the second membrane. In another embodiment, the drug cavity port includes a backing plate to prevent puncture of the first membrane or the second membrane. The backing plate is made from a magnetic material in some embodiments. In another embodiment, he backing plate is mounted as a component of the drug cavity port such that the backing plate does not contact the non-permeable shell. In one embodiment, the backing plate is mounted to an outer surface of the non-permeable shell.

In another embodiment, the device has a plurality of drug cavity ports that provide access to the drug cavity. For example, in one embodiment, a first drug cavity port in the plurality of drug cavity ports connects to the second membrane by way of a drug cavity port tube such that the first drug cavity port is remote from the second membrane, and a second drug cavity port in the plurality of drug cavity ports is formed on a surface of the second membrane. In another embodiment, the expansion cavity port connects to the expansion cavity by way of an expansion cavity port tube such that the expansion cavity port is remote from the non-permeable shell. In one embodiment, an outer surface of the expansion cavity port tube forms a seal with an opening in the second membrane.

In another embodiment, the second membrane surrounds the first membrane to form the drug cavity in between the first membrane and the second membrane. In another embodiment, the second membrane is semi-permeable such that the therapeutic substance is able to exit the drug cavity into the patient via the second membrane. In an alternative embodiment, the first membrane surrounds the second membrane such that the drug cavity is formed within the expansion cavity. In such an embodiment, the device can include a permeable channel that extends from the drug cavity to the first membrane. End walls of the permeable channel are at least semi-permeable to allow the therapeutic solution to move from the drug cavity into the patient. A sidewall of the permeable channel is non-permeable to prevent the therapeutic solution from entering the expansion cavity as the therapeutic solution travels from the drug cavity to the patient.

An illustrative method of making an expander and drug delivery device includes forming an expansion cavity with a first membrane, where the first membrane is a non-permeable shell. The method also includes mounting an expansion cavity port in communication with the expansion cavity. The expansion cavity port provides access to the expansion cavity, and the non-permeable shell is flexible such that the non-permeable shell inflates in response to insertion of a substance into the expansion cavity via the expansion cavity port. The method also includes forming a drug cavity with a second membrane such that the drug cavity is adjacent to the expansion cavity. The method further includes mounting a drug cavity port in communication with the drug cavity, wherein the drug cavity port is sized to receive a therapeutic solution for delivery to a patient.

In one embodiment, forming the drug cavity comprises positioning the second membrane external to the first membrane such that the drug cavity is formed in between the first membrane and the second membrane. In another embodiment, forming the drug cavity comprises positioning the second membrane within the first membrane such that the drug cavity is formed within the expansion cavity. In another embodiment, the method includes determining a size and shape of a breast cavity of the patient, and where a size and shape of the expansion cavity and the drug cavity of the expander and drug delivery device are based on the determined size and shape of the breast cavity.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts an expander and drug delivery device with external ports in accordance with a first illustrative embodiment.

FIG. 1B shows an expansion and drug delivery device with internal ports in accordance with a second illustrative embodiment.

FIG. 1C depicts an expander and drug delivery device with an external port for the expansion chamber and an internal port for the drug chamber in accordance with a third illustrative embodiment.

FIG. 1D depicts an expansion and drug delivery device with an internal port for the expansion chamber and an external port for the drug chamber in accordance with a fourth illustrative embodiment.

FIG. 1E is a cross-sectional view depicting a one way spring valve in a closed position in accordance with an illustrative embodiment.

FIG. 1F is a cross-sectional view depicting the one way spring valve in an open position in accordance with an illustrative embodiment.

FIG. 2A depicts an expansion and drug delivery device with an expansion chamber external to the drug chamber in accordance with a first illustrative embodiment.

FIG. 2B depicts an expansion and drug delivery device with an expansion chamber external to the drug chamber in accordance with a second illustrative embodiment

FIG. 2C depicts an expansion and drug delivery device with an expansion chamber external to the drug chamber in accordance with a third illustrative embodiment.

FIG. 2D depicts an expansion and drug delivery device with an expansion chamber external to the drug chamber in accordance with a fourth illustrative embodiment.

FIG. 3 depicts an expansion and drug delivery device that includes a catheter in accordance with an illustrative embodiment.

FIG. 4 depicts an expander and drug delivery device with multiple outer shell port sites in accordance with an illustrative embodiment.

FIG. 5 depicts an expander and drug delivery device with multiple inner shell ports in accordance with an illustrative embodiment.

FIG. 6 depicts an expansion and drug delivery device that includes a plurality of suture tabs in accordance with an illustrative embodiment.

### DETAILED DESCRIPTION

Implant-based breast reconstruction refers to a reconstructive technique that is used to recreate a breast after mastectomy. Common problems encountered during implant-based reconstruction procedures include pain experienced as a result of the expansion, infection from the introduction of a foreign device (i.e., expander) into the patient's body, and pauses in reconstruction due to the need for ongoing chemotherapy or other treatment.

Described herein is an improved tissue expander device that enables local drug delivery via a second port connected to a second (outer) semi-permeable lumen that can accept a drug or other substance in liquid form. Drugs that can be delivered through the proposed device include a local anesthetic (to decrease pain associated with the expansion procedure), antibiotics (to help prevent or cure infections that result from introduction of the device into the patient), cancer-targeting therapeutics to help prevent local cancer recurrence, etc.

In an illustrative embodiment, the proposed device includes a tissue expander, which is made up of a non-permeable shell that is formed from silicone or another biocompatible non-permeable material. The non-permeable shell forms a cavity (or lumen or chamber) that is inflatable. In general, the chamber is inflated with a substance such as air or a saline solution. The non-permeable shell can be inflated via a remote port site that connects an interior of the non-permeable shell by way of hollow tubing. The proposed device also includes an outer shell that surrounds the non-permeable shell. The outer shell is designed to be filed with a solution that includes a drug or other therapeutic, as discussed herein. In an illustrative embodiment, the outer shell is formed as a semi-permeable membrane that allows the diffusion of whatever solution is contained in the outer shell lumen (or chamber or cavity). The outer shell has at least one second port site that is used to deliver the solution (e.g., via percutaneous drug delivery).

In one embodiment, the second port site for the outer shell can be remote (i.e., similar to the inner shell) such that the second port site is connected to the cavity of the outer shell via hollow tubing. Alternatively, the second port site can be built into the outer shell as a one-way valve that allows the introduction of solution (e.g., drug) but prevents sudden, uncontrolled escape of the solution. In an embodiment in which the second port site is built directly into the outer shell, the second port site can have a backing plate that helps to both locate the second port site percutaneously, as well as prevent through-and-through puncture of the inner shell during introduction of a solution into the second port site. In one embodiment, the backing plate can be formed from a magnetic material to assist with location identification and/or manipulation of the implant.

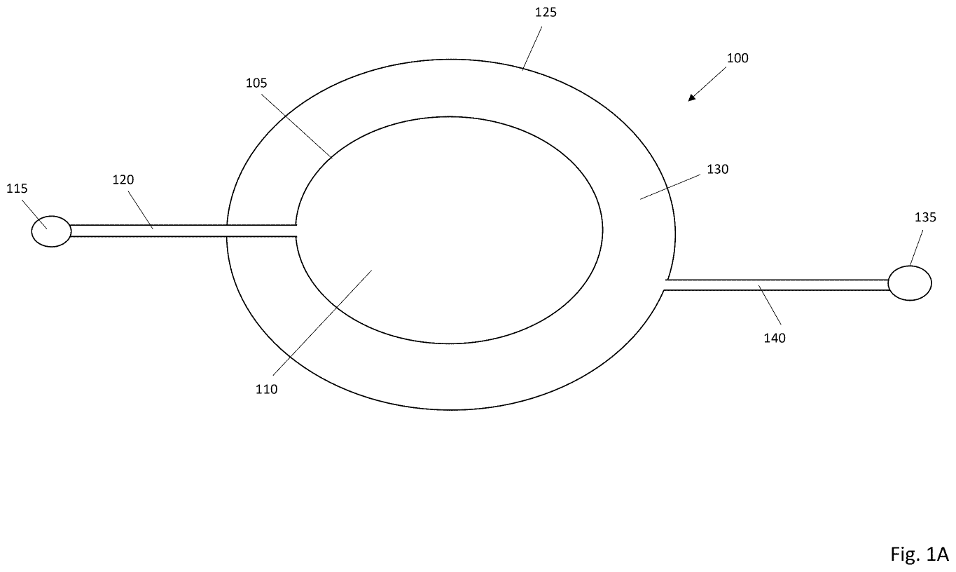

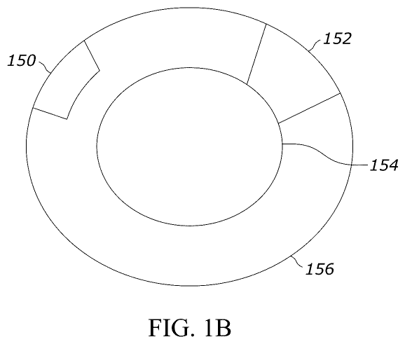

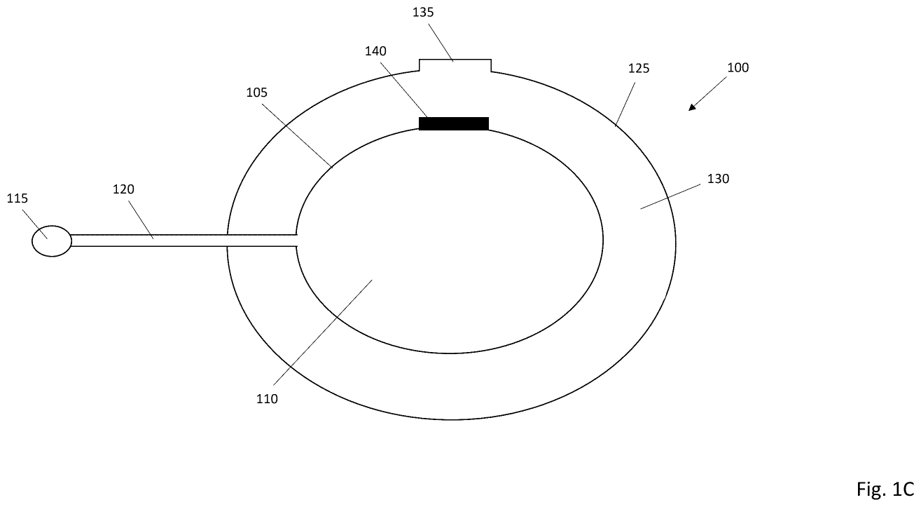

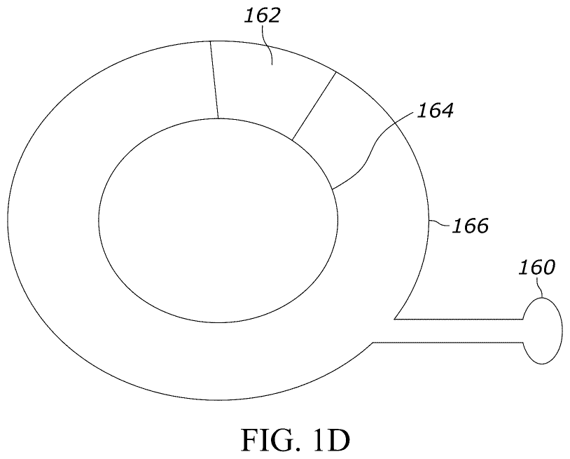

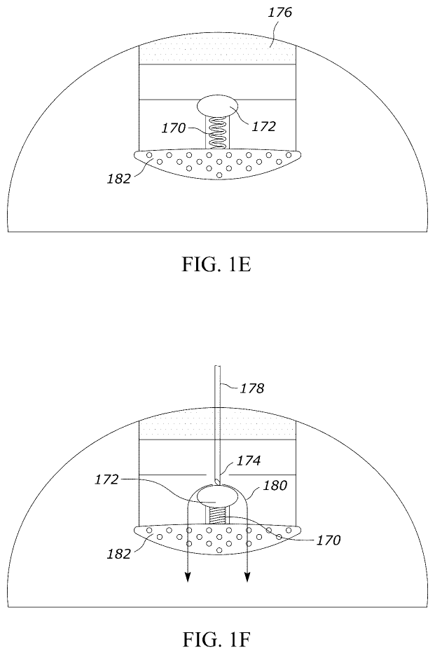

FIG. 1A depicts an expander and drug delivery device 100 with external ports in accordance with a first illustrative embodiment. An inner portion of the device 100 includes a non-permeable membrane 105 that forms an expansion chamber (or cavity) 110. The non-permeable membrane 105 is formed from a material that is able to expand, such that the expansion chamber 110 is able to be inflated and expand in size. In one embodiment, the non-permeable membrane 105 is formed from silicon. In an illustrative embodiment, the expansion chamber 110 is designed to receive a substance (e.g., air, water, saline, etc.) that causes the inflation and expansion. The substance is delivered to the expansion chamber 110 via an expansion chamber port 115. As shown, the expansion chamber port 115 is connected to the expansion chamber 110 with an expansion chamber port tube 120, which is a hollow tube that allows the substance to pass from the expansion chamber port 115 into the expansion chamber 110. The expansion chamber port 115 and the expansion chamber port tube 120 provide continuous remote access into the expansion chamber 110.

The device 100 of FIG. 1A also includes an outer shell 125 that surrounds the non-permeable membrane 105. The outer shell 125 forms a drug chamber (or cavity) 130 that is external to and that surrounds the non-permeable membrane 105. In an illustrative embodiment, the outer shell 125 is formed from a semi-permeable material that allows a drug or other substance introduced into the drug cavity 130 to leave the outer shell 125 to provide a therapeutic effect for the patient. A rate at which the introduced drug/therapeutic leaves the drug chamber 130 through the outer shell 125 can be controlled by controlling a thickness of the outer shell 125 and/or the type of semi-permeable material from which the outer shell 125 is made.

In an illustrative embodiment, the outer shell can be composed of either a biodegradable or non-biodegradable polymer. Examples of such polymers include polysiloxanes, polyethylenevinylacetate, polyurethanes, polylactic acid, polyglycolic acid, polylacticcoglycolic acid, polycaprolactone, cellulose, chitosan, and silk. Alternatively, both shells could be composed of non-permeable polymer so that the outer shell can be designated for expansion while the inner shell is designated for drug delivery. In such an embodiment, the inner shell would have a permeable channel that allows drug diffusion from the inner shell, as described in more detail below. Thickness of shell membranes can range from ˜0.1 to ˜10 millimeters (mm) thick. Drug dispersion can be controlled by back pressure created by filling the inner or outer reservoir of the expander device with saline (whichever lumen is designated for expansion), in addition to membrane/channel porosity. Membrane/channel porosity can be either nanoporous, microporous, macroporous, or ultraporous. In one embodiment, either an internal or external pump device can be used to help control the flow rate.

The drug/therapeutic is delivered to the drug chamber 130 via an outer shell port 135 that connects to the drug chamber 130 via an outer shell port tube 140. The outer shell port 135 and the outer shell port tube 140 provide continuous access into the drug chamber 130. The outer shell port tube 140 is a hollow tube that allows the drug or therapeutic to pass from the outer shell port 135 into the drug chamber 130. The introduced drug/therapeutic is unable to pass into the expansion chamber 110 due to the non-permeable nature of the non-permeable membrane 105 that forms the expansion chamber 110. As shown, the expansion chamber port tube 120 passes through the outer shell 125 to provide remote access for inflation of the expansion chamber 110. The outer shell 125 forms a seal around an outer surface of the expansion chamber port tube 120 such blood is unable to enter the drug cavity 130 at the outer shell 125 expansion chamber port tube 120 interface, and also so that a drug from the drug cavity 130 is unable to exit into the patient's bloodstream at the interface. The seal can be formed through integral construction, use of an adhesive (e.g., glue) at the interface, or through a friction fit, depending on the implementation. In an illustrative embodiment, the expansion chamber port 115 and the outer shell port 135 are external ports that can be accessed external to the patient (i.e., non-invasively).

FIG. 1B shows an expansion and drug delivery device with internal ports in accordance with a second illustrative embodiment. Specifically, FIG. 1B shows a nonpermeable membrane 154 that forms the expansion chamber (or inner lumen) and a permeable membrane 156 that forms the drug chamber (or outer lumen). In the embodiment of FIG. 1B, a first internal port 152 is used to access the expansion chamber formed by the nonpermeable membrane 154 and a second internal port is used to access the drug chamber formed by the permeable membrane 156.

FIG. 1C depicts an expander and drug delivery device 100 with an external port for the expansion chamber and an internal port for the drug chamber in accordance with a third illustrative embodiment. Similar to the device of FIG. 1A, an inner portion of the device 100 includes a non-permeable membrane 105 that forms an expansion chamber (or cavity) 110. The non-permeable membrane 105 is again formed from a material that is able to expand, such that the expansion chamber 110 is able to be inflated and expand in size. In an illustrative embodiment, the expansion chamber 110 is designed to receive a substance (e.g., air, water, saline, etc.) that causes the inflation and expansion such that the device 100 operates as an expander to slowly stretch the covering skin. The substance is delivered to the expansion chamber 110 via an expansion chamber port 115. The expansion chamber port 115 is connected to the expansion chamber 110 with an expansion chamber port tube 120, which is a hollow tube that allows the substance to pass from the expansion chamber port 115 into the expansion chamber 110. In an illustrative embodiment, the expansion chamber port 115 is an external port that can be accessed external to the patient (i.e., non-invasively).

The device 100 of FIG. 1C also includes an outer shell 125 that surrounds the non-permeable membrane 105. The outer shell 125 forms a drug chamber (or cavity) 130 that is external to and that surrounds the non-permeable membrane 105. In an illustrative embodiment, the outer shell 125 is formed from a semi-permeable material that allows a drug or other substance introduced into the drug cavity 130 to leave the outer shell 125 to provide a therapeutic effect for the patient. A rate at which the introduced drug/therapeutic leaves the drug chamber 130 through the outer shell 125 can be controlled by controlling a thickness of the outer shell 125 and/or the type of semi-permeable material from which the outer shell 125 is made. In another illustrative embodiment, any of the ports

The drug/therapeutic is delivered to the drug chamber 130 via an outer shell port 135 that connects directly to the drug chamber 130. As such, a syringe or other delivery device can pass through the outer shell port 135 to provide direct delivery of the drug or therapeutic into the drug chamber 130. The outer shell port 135 can be internal to the patient (i.e., invasively accessed). The outer shell port 135 can be in the form of a one-way valve. The one way valve can be formed from a spring mechanism that can be compressed with needle insertion and then reloads upon release to seal the port. The one-way valve can also be made from a polymer matrix, such as silicone, that is of a consistency that allows it to reseal itself upon needle removal. In some embodiments, the expansion chamber port can be made from the same mechanism as the outer shell port. The introduced drug/therapeutic is unable to pass into the expansion chamber 110 due to the non-permeable nature of the non-permeable membrane 105 that forms the expansion chamber 110.

FIG. 1E is a cross-sectional view depicting a one way spring valve in a closed position in accordance with an illustrative embodiment. FIG. 1F is a cross-sectional view depicting the one way spring valve in an open position in accordance with an illustrative embodiment. As shown, the one way valve includes a spring 170 with a stop 172 mounted to an end of the spring 170. The stop 172 blocks an opening in 174 in the valve and in the position of FIG. 1E the stop 172 prevents fluid from entering or leaving the expander device. An outer layer of the one way spring valve is formed from a self-sealing polymer 176 that has the ability to reseal itself after being punctured by a tube, syringe, etc. FIG. 1F shows a needle (or tube) 178 passing through the self-scaling polymer 176 and contacting the stop 172. Placing pressure on the stop 172 via the needle 178 causes the spring 170 to compress, which removes the stop 172 from the opening 174 and allows fluid 180 to flow from the needle 178 through a semi-rigid porous layer 182 and into the chamber (i.e., either the expansion chamber or the drug chamber). Upon removal of the needle 178, the spring 170 expands back to its extended position (FIG. 1E) and causes the stop 172 to reseal the opening 174, thereby preventing the escape of fluid through the port/valve. Additionally, removal of the needle 178 allows the self-sealing polymer 176 to reseal, thereby preventing unwanted material from entering the valve/port. The one way spring valve can be used to implement any of the ports described herein.

The outer shell port 135 also includes a backing plate 140 which prevents a syringe/needle that enters the outer shell port 135 from being able to puncture the non-permeable membrane 105. The backing plate 140 can be made from any biocompatible material that is resistant to needle puncture, such as metal, hard plastic, hard rubber, etc. In an illustrative embodiment, the backing plate 140 can be made from a magnetic material to assist with location determination of the outer shell port 135 using an imaging system and/or manipulation of the implant. In one embodiment, the backing plate 140 can be formed as a component of the outer shell port 135. Alternatively, the backing plate 140 may be mounted to an outer surface of the non-permeable membrane 105.

FIG. 1D depicts an expansion and drug delivery device with an internal port 162 for the expansion chamber and an external port 160 for the drug chamber in accordance with a fourth illustrative embodiment. As noted, FIGS. 1B and 1D depict an internal port to access the expansion chamber, which is positioned within the drug chamber in this embodiment. In such an embodiment, the internal port 162 can be an extended port that is positioned within the drug chamber and that traverses a distance between the non-permeable membrane 164 that forms the expansion chamber and the permeable membrane 166 that forms the drug chamber. This extended port ensures that fluid used to fill the expansion chamber does not enter the drug chamber.

In another illustrative embodiment, any of the ports described herein can be two-way ports such that fluid can also be removed from the implant site. For example, one or more ports can be used to remove fluid external to the implant as in the case of seromas that can form around implants or expander device. Additionally, in some embodiments, the overall shape of the expansion and drug delivery device can be determined by a shape of the defect cavity which is to be reconstructed. For example, a size (e.g., volume) and overall shape of the breast cavity after a lumpectomy can be determined using imaging, and a custom mold can be fabricated based on the size/shape of the cavity and used to form the expansion and drug delivery device such that is custom made for a given patient.

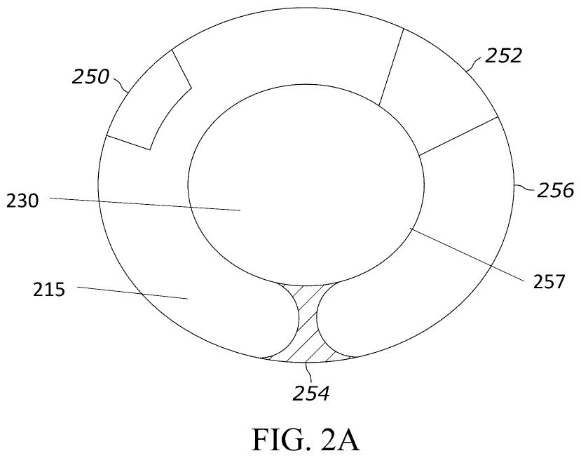

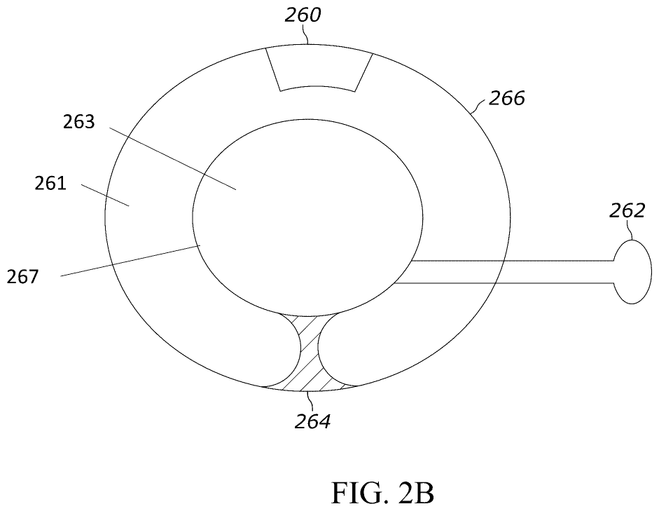

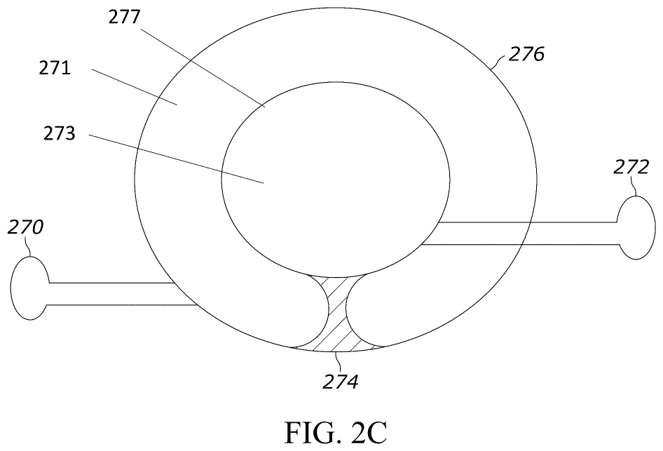

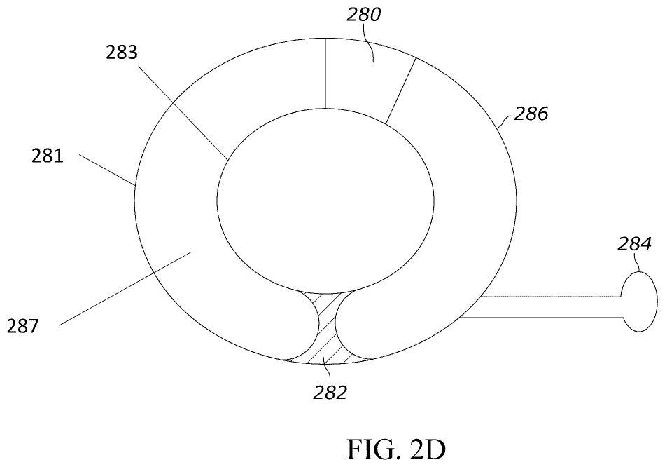

FIGS. 2A-2D depict various embodiments in which the non-permeable membrane that forms the expansion chamber is external to the drug chamber. FIG. 2A depicts an expansion and drug delivery device with an expansion chamber 215 external to the drug chamber 230 in accordance with a first illustrative embodiment. The expansion chamber 215 is formed by a non-permeable membrane 256 and the drug chamber 230 is primarily (i.e., except for the interface at a permeable channel 254) formed by a non-permeable membrane 257. In FIG. 2A, there is an internal port 252 for the expansion chamber 215, an internal port 250 for the drug chamber 230, and the permeable channel 254 is formed from the drug chamber 230 to an exterior of the expansion chamber such that the drug/therapeutic can be delivered to the patient. The interface between the permeable channel 254 and the drug chamber 230 is at least semi-permeable such that the drug, etc. can exit through the drug chamber 230 and proceed through the permeable channel 254 into the bloodstream of the patient.

More specifically, a first end wall of the permeable channel 254 is permeable (or semi-permeable) and forms an interface at the non-permeable membrane 257 that forms the drug chamber 230. This interface allows the drug, etc. to exit the otherwise impermeable drug chamber 230. A second end wall of the permeable channel is permeable (or semi-permeable) and forms an interface at the non-permeable membrane 256 that forms the expansion chamber 215. This interface allows the drug, etc. to exit the permeable channel 254 and enter the bloodstream of the patient. In an illustrative embodiment, a sidewall of the permeable channel 254 is non-permeable such that the drug, etc. is unable to pass from the permeable channel 254 into the expansion chamber 215.

FIG. 2B depicts an expansion and drug delivery device with an expansion chamber 261 external to the drug chamber 263 in accordance with a second illustrative embodiment. The expansion chamber 261 is formed by a non-permeable membrane 266 and the drug chamber 263 is primarily (i.e., except for the interface at a permeable channel 264) formed by a non-permeable membrane 267. In FIG. 2B, there is an internal port 260 for the expansion chamber, an external port 262 for the drug chamber, and the permeable channel 264 is formed from the drug chamber to an exterior of the expansion chamber such that the drug/therapeutic can be delivered to the patient. The permeable channel 264 can have the same configuration/structure as the permeable channel 254 described with reference to FIG. 2A.

FIG. 2C depicts an expansion and drug delivery device with an expansion chamber 271 external to the drug chamber 273 in accordance with a third illustrative embodiment. The expansion chamber 271 is formed by a non-permeable membrane 276 and the drug chamber 273 is primarily (i.e., except for the interface at a permeable channel 274) formed by a non-permeable membrane 277. In FIG. 2C, there is an external port 270 for the expansion chamber, an external port 272 for the drug chamber, and the permeable channel 274 is formed from the drug chamber to an exterior of the expansion chamber such that the drug/therapeutic can be delivered to the patient. The permeable channel 274 can have the same configuration/structure as the permeable channel 254 described with reference to FIG. 2A.

FIG. 2D depicts an expansion and drug delivery device with an expansion chamber 281 external to the drug chamber 283 in accordance with a fourth illustrative embodiment. The expansion chamber 281 is formed by a non-permeable membrane 286 and the drug chamber 283 is primarily (i.e., except for the interface at a permeable channel 282) formed by a non-permeable membrane 287. In FIG. 2D, there is an internal port 280 for the drug chamber, an external port 284 for the expansion chamber, and the permeable channel 282 is formed from the drug chamber to an exterior of the expansion chamber such that the drug/therapeutic can be delivered to the patient. The permeable channel 282 can have the same configuration/structure as the permeable channel 254 described with reference to FIG. 2A.

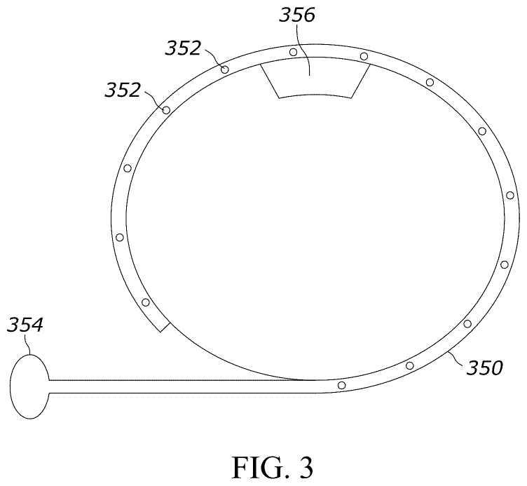

FIG. 3 depicts an expansion and drug delivery device that includes a catheter 350 in accordance with an illustrative embodiment. In the embodiment shown, the device includes a non-permeable membrane 351 that forms an expansion chamber 353, and an internal port 356 that is used for expansion of the expansion chamber. In an alternative embodiment the expansion chamber may have an external port instead of the internal port shown. The catheter 350 is mounted on an exterior surface of the non-permeable membrane 351 that forms the expansion chamber 353. The catheter includes a plurality of holes/openings 352 that allow for immediate drug delivery. In an alternative embodiment, the holes of the catheter may be covered with a permeable or semi-permeable membrane that allows for a slower delivery of the drugs/therapeutics. The drugs/therapeutics can be delivered to the catheter via the depicted external port 354. In one embodiment, the catheter can be in the form of a thin separate tube that mounts along a lower circumference or the full circumference of the expansion chamber. Alternatively, the catheter may be replaced by a thin bladder mounted along the lower portion or full circumference of the expansion chamber. The thin bladder can be made from a permeable membrane such that openings are not used.

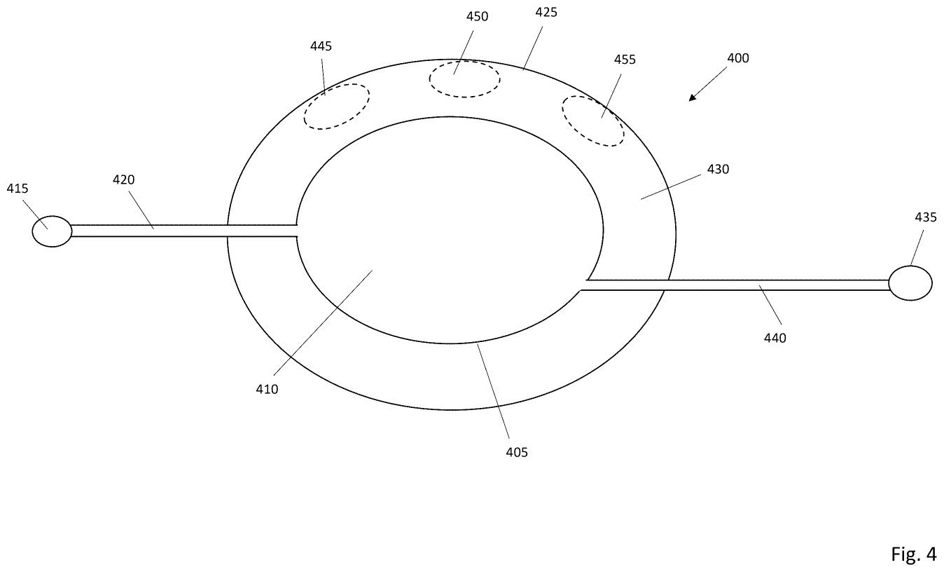

FIG. 4 depicts an expander and drug delivery device 400 with multiple outer shell port sites in accordance with an illustrative embodiment. Similar to the devices of FIGS. 1 and 2, the expander and drug delivery device 400 includes a non-permeable membrane 405 that forms an expansion chamber 410, and a (semi-permeable) outer shell 425 that forms a drug chamber 430. The expander and drug delivery device 400 also includes a first expansion chamber port 415 (connected to the expansion chamber 410 via a first expansion chamber port tube 420) and a second expansion chamber port 435 (connected to the expansion chamber 410 via a second expansion chamber port tube 440). In an illustrative embodiment, the first expansion chamber port 415 and the second expansion chamber port 435 are external ports that can be accessed external to the patient (i.e., non-invasively). The use of multiple ports allows for multiple access points. This can be helpful if the device rotates or flips in the patient. In alternative embodiments, fewer or additional expansion chamber ports may be used. For example, in some embodiments, both the expansion chamber and the drug chamber may include a plurality of ports.

FIG. 4 also depicts a first outer shell port location 445, a second outer shell port location 450, and a third outer shell port location 455. In an illustrative embodiment, only one of the outer shell port locations will include an outer shell port. Alternatively, a plurality of the outer shell port locations can include an outer shell port such that the device 400 includes multiple outer shell ports formed directly in the outer shell 425. While three outer shell port locations are shown for illustrative purposes, it is to be understood that fewer or additional outer shell port locations may be used in alternative embodiments. Additionally, an outer shell port can be placed at any location on the outer shell 425, and is not limited to the specific regions depicted in FIG. 4.

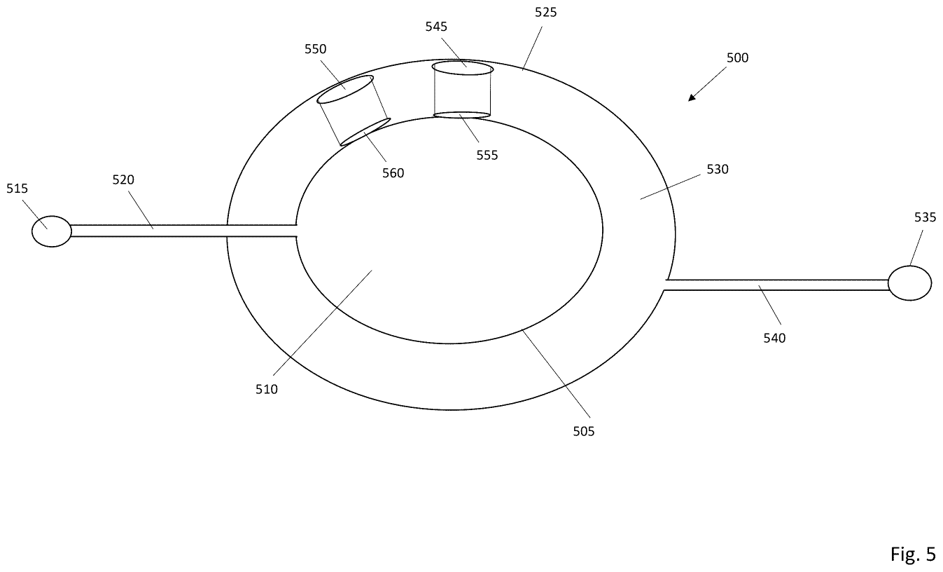

FIG. 5 depicts an expander and drug delivery device 500 with multiple inner shell ports in accordance with an illustrative embodiment. Similar to the devices of FIGS. 1, 2, and 4, the expander and drug delivery device 500 includes a non-permeable membrane 505 that forms an expansion chamber 510, and a (semi-permeable) outer shell/membrane 525 that forms a drug chamber 530. The expander and drug delivery device 500 also includes an expansion chamber port 515 (connected to the expansion chamber 510 via an expansion chamber port tube 520) and a first outer shell port 535 (connected to the drug chamber 530 via an outer shell port tube 540). In an illustrative embodiment, the expansion chamber port 515 and the first outer shell port 535 are external ports that can be accessed external to the patient (i.e., non-invasively). In alternative embodiments, fewer or additional external expansion chamber ports and/or outer shell ports may be used.

FIG. 5 also depicts a second outer shell port 545 and a third outer shell port 550, each of which is formed in the surface of the outer shell 525. In alternative embodiments, fewer or additional outer shell ports may be formed in the surface of the outer shell. The second outer shell port 545 has a first backing plate 555 to prevent puncture of the expansion chamber 410 and the third outer shell port 550 has a second backing plate 560 to prevent such a puncture. The backing plates can be incorporated as a component of the port in an illustrative embodiment. Alternatively, the backing plates may be attached to a portion of the non-permeable membrane 505 that lies below/behind the outer shell ports.

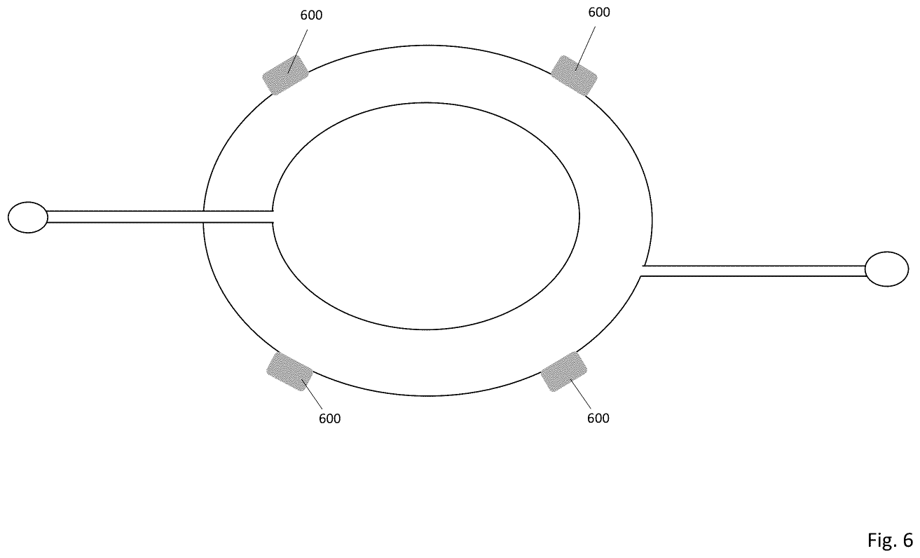

FIG. 6 depicts an expansion and drug delivery device that includes a plurality of suture tabs 600 in accordance with an illustrative embodiment. The suture tabs allow fixation of the expander to surrounding tissues. While four suture tabs are shown, it is to be understood that fewer or additional suture tabs (e.g., 2, 3, 5, 6, etc.) may be used in alternative embodiments. Additionally, the suture tabs are not limited to the mounting positions shown in the figure, and may be positioned in any location on the outer surface of device. The suture tabs can be used with any of the various embodiments described herein.

As discussed herein, the proposed device can be used to assist with breast reconstruction following a mastectomy or other breast procedure. However, the device is not limited to use during breast reconstruction. The proposed device can also be used in other types of procedures such as burn reconstruction, reconstruction of pediatric congenital defects, other types of cancer reconstruction, and general trauma reconstruction ranging anywhere from the scalp, head, neck, chest, abdomen, back, or extremities.

The proposed device enables local drug delivery at a site of tissue expansion, which can be beneficial when systemic delivery is undesired due to a lessened effect of the drug and/or an increased risk of systemic side effects. As discussed, the device can be used for delivery of local anesthetic to prevent pain with tissue expansion, for antibiotic delivery to prevent infection, for therapeutic drug delivery to prevent cancer recurrence, etc. The device allows for repeated local drug delivery, avoids liver drug metabolism (which can be seen with oral drug delivery), and also allows controlled release of a local drug via the semi-permeable membrane such that controlled diffusion or elution can be achieved.

The proposed device has the potential to not only improve the tissue expansion process for patients by enabling a route of local anesthetic delivery to decrease pain, but also has the potential to improve outcomes both by allowing drug delivery such as antibiotics for infection prevention and also local therapeutics for cancer treatment. One of the most common reasons for tissue expander failure is infection. Usually, this is treated with systemic antibiotics, often via intravenous antibiotics. Such treatment requires admission to a hospital for several days and expensive interventions such as the placement of a venous catheter for IV antibiotic delivery at the patient's home. By enabling the local delivery of antibiotic, this could prevent the need for hospital admission or additional procedures when these devices do get infected. This could also be used for preventative purposes by delivering antibiotic locally until the incisions have healed to prevent infection from developing in the first place. The chemotherapeutic delivery could help local delivery of key drugs not only in breast cancer but in the larger class of all soft tissue cancers, anywhere in the body.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. An expander and drug delivery device, comprising:
a first membrane, wherein the first membrane is a non-permeable shell that forms an expansion cavity,
an expansion cavity port that provides access to the expansion cavity, wherein the non-permeable shell is flexible such that the non-permeable shell inflates in response to insertion of a substance into the expansion cavity via the expansion cavity port;
a second membrane that forms a drug cavity adjacent to the expansion cavity; and
a drug cavity port that connects to the drug cavity and receives a therapeutic solution for delivery to a patient.

2. The device of claim 1, wherein the drug cavity port connects to the drug cavity by way of a drug cavity port tube such that the drug cavity port is remote from the second membrane.

3. The device of claim 1, wherein the drug cavity port is formed on a surface of the second membrane.

4. The device of claim 3, wherein the drug cavity port includes a backing plate to prevent puncture of the first membrane or the second membrane.

5. The device of claim 4, wherein the backing plate is made from a magnetic material.

6. The device of claim 4, wherein the backing plate is mounted as a component of the drug cavity port such that the backing plate does not contact the non-permeable shell.

7. The device of claim 4, wherein the backing plate is mounted to an outer surface of the non-permeable shell.

8. The device of claim 1, further comprising a plurality of drug cavity ports that provide access to the drug cavity.

9. The device of claim 8, wherein a first drug cavity port in the plurality of drug cavity ports connects to the second membrane by way of a drug cavity port tube such that the first drug cavity port is remote from the second membrane, and wherein a second drug cavity port in the plurality of drug cavity ports is formed on a surface of the second membrane.

10. The device of claim 1, wherein the expansion cavity port connects to the expansion cavity by way of an expansion cavity port tube such that the expansion cavity port is remote from the non-permeable shell.

11. The device of claim 10, wherein an outer surface of the expansion cavity port tube forms a seal with an opening in the second membrane.

12. The device of claim 1, wherein the second membrane surrounds the first membrane to form the drug cavity in between the first membrane and the second membrane.

13. The device of claim 12, wherein the second membrane is semi-permeable such that the therapeutic substance is able to exit the drug cavity into the patient via the second membrane.

14. The device of claim 1, wherein the first membrane surrounds the second membrane such that the drug cavity is formed within the expansion cavity.

15. The device of claim 14, further comprising a permeable channel that extends from the drug cavity to the first membrane.

16. The device of claim 15, wherein end walls of the permeable channel are at least semi-permeable to allow the therapeutic solution to move from the drug cavity into the patient.

17. The device of claim 15, wherein a sidewall of the permeable channel is non-permeable to prevent the therapeutic solution from entering the expansion cavity as the therapeutic solution travels from the drug cavity to the patient.

18. A method of making an expander and drug delivery device, the method comprising:
forming an expansion cavity with a first membrane, wherein the first membrane is a non-permeable shell;
mounting an expansion cavity port in communication with the expansion cavity, wherein the expansion cavity port provides access to the expansion cavity, and wherein the non-permeable shell is flexible such that the non-permeable shell inflates in response to insertion of a substance into the expansion cavity via the expansion cavity port;
forming a drug cavity with a second membrane such that the drug cavity is adjacent to the expansion cavity; and
mounting a drug cavity port in communication with the drug cavity, wherein the drug cavity port is sized to receive a therapeutic solution for delivery to a patient.

19. The method of claim 18, wherein forming the drug cavity comprises positioning the second membrane external to the first membrane such that the drug cavity is formed in between the first membrane and the second membrane.

20. The method of claim 18, further comprising determining a size and shape of a breast cavity of the patient, and wherein a size and shape of the expansion cavity and the drug cavity of the expander and drug delivery device are based on the determined size and shape of the breast cavity.
